(19) United States
(12) Patent Application Publication
(10) Pub. No.: US 2012/0037162 A1
(43) Pub. Date: Feb. 16, 2012

(54) VENTILATORY ASSISTANCE SYSTEM

(76) Inventor: Georges Boussignac, Antony (FR)

(21) Appl. No.: 13/191,036

(22) Filed: Jul. 26, 2011

(30) Foreign Application Priority Data
Aug. 12, 2010 (FR) 10/03343

(51) Int. Cl. A61M 16/04

(52) U.S. Cl. 128/207.14

(57) ABSTRACT

The present method, system and device relate to a ventilatory assistance system comprising a ventilatory assistance device, which comprises a tube on which a tubular nozzle is integrally mounted, and a respiratory gas supply duct able to connect said tubular nozzle to a source of respiratory gas. The present ventilatory assistance system further comprises obstacle forming means counteracting against assembling a connecting member of an external medical device to said tubular nozzle, when the latter is connected to said source of respiratory gas by means of said supply duct.

### CROSS-REFERENCE TO RELATED APPLICATION(S)

This application claims priority to French application No. 10/03343, filed Aug. 12, 2010, entitled A VENTILATORY ASSISTANCE SYSTEM, the contents of which are expressly incorporated herein by reference.

### FIELD OF ART

The present method, system and device provide a ventilatory assistance system, more specifically intended for reanimating a patient in a cardiac arrest condition.

### BACKGROUND

Ventilatory assistance systems are already known, comprising:
one ventilatory assistance device comprising:
   one tube, forming a main channel and being intended for being connected, through its distal end, to an airway of a patient so that the main channel connects, outside, the patient's breathing system;
   auxiliary channels opening into the main channel upstream the distal end thereof; and
   tubular nozzle integrally mounted, tightly, at the proximal end of the tube and being able to be connected to an artificial respirator, and
one respiratory gas supply duct able to connect the tubular nozzle to the source of respiratory gas so as to supply respiratory gas to the auxiliary channels.

Thus, in the case where a patient is in a cardiac arrest condition, it is possible to ventilate his/her lungs with respiratory gas using such a ventilatory assistance system. This system can be implemented, as known, as follows:
first, a part of the tube is introduced in the patient's trachea. The ventilatory assistance device is subsequently connected, at the level of its tubular nozzle, to a source of respiratory as (for instance a bottle of oxygen), by means of the supply duct. The patient is then ventilated with oxygen originating from said source, after having been transferred via the supply duct and the longitudinal channels. Upon the continuous insufflation of oxygen, an auxiliary nursing staff exerts alternating compressions and decompressions on the patient's ribcage allowing a ventilation of the latter (still referred to as a passive oxygenation); and
after the heart is working again, the supply duct is disconnected from the tubular nozzle (and hence, from the ventilatory assistance device). An artificial respirator can then be connected directly to the tubular nozzle of the ventilatory assistance device.

However, within the rush of the situation, errors could be made by the auxiliary nursing staff. In particular, it happens that the tubular nozzle of the tube introduced in the patient's trachea is inadvertently connected to an artificial respirator before the supply duct is disconnected from said nozzle.

In this latter case, the patient's lungs are ventilated with oxygen both originating from the external source and from the artificial respirator. Because the artificial respirator is a closed system, this leads to an increase of pressure inside said lungs able to compromise the already impaired patient's health.

### SUMMARY

The present method, system and device aim at overcoming such a drawback and, more particularly, avoiding the occurrence of an overpressure in the breathing system of a patient subject to a continuous insufflation of respiratory gas.

To this end, according to the present method, system and device, the ventilatory assistance system comprising:
a ventilatory assistance device comprising:
   a tubular body, forming a main channel and being intended for being connected, through its distal end, to an airway of a patient so that the main channel connects, outside, the patient's breathing system;
   at least one auxiliary channel opening into the main channel upstream distal end thereof; and
   a tubular nozzle integrally mounted, tightly, at the proximal end of the tubular body; and
   a respiratory gas supply duct able to connect said tubular nozzle to a source of respiratory gas, so as to supply respiratory gas to said auxiliary channel.

The present system is remarkable in that it comprises obstacle forming means, counteracting against assembling a connecting member of an external medical device to said tubular nozzle, when the latter is connected to said source of respiratory gas by means of said supply duct.

Thus, thanks to the present method, system and device, no external medical device (for instance an artificial respirator) could be connected to the tubular nozzle, in the case where the latter is already connected to a source of respiratory gas. In the case of a cardiac arrest with a patient, the patient's ventilation, achieved through the continuous insufflation of respiratory gas combined to compressions and decompressions carried out by the auxiliary nursing staff generating a positive intra thoracic pressure (during the compressions) and a negative intra thoracic pressure (during the decompressions) allowing to achieve a better hemodynamics, is thereby fully secured. There is indeed no risk of an overpressure occurring inside the patient's airways, such an overpressure being caused for instance by an additional respiratory gas supply originating from a closed system type external medical device (for instance an artificial respirator).

Moreover, the present method, system and device allow to simplify managing a patient being in a cardiac arrest condition, so that only one person can attend the latter.

Moreover, said obstacle forming means are advantageously mounted at the distal end of said supply duct.

Thus, after the supply duct of the tubular nozzle has been disconnected (for instance when the heart is working again) causing the continuous insufflation of respiratory gas to stop, the tubular nozzle of the ventilatory assistance device is released from the obstacle forming means and is able, from now on, to be connected to an external medical device. In such a case, the obstacle forming means are removable with respect to the tubular nozzle of the ventilatory assistance device. They only cooperate with the tubular nozzle when it is connected to the supply duct.

It is understood that, alternatively, the obstacle forming means could be mounted directly on the ventilatory assistance device, or even be independent and removable from the latter and from the supply duct.

In an embodiment according to the present method, system and device, said obstacle forming means comprise an opened groove defining a housing able to receive at least one part of said tubular nozzle, when the latter is connected to said source of respiratory gas through said supply duct. Alternatively, it could obviously be contemplated that said obstacle forming means comprise a closed groove.

Thus, when at least one part of the tubular nozzle is housed in the groove, the latter forms an overthickness on the covered part of the external side wall of said tubular nozzle, preventing any connection to an external medical device, during the insufflation of respiratory gas originating from said source.

Moreover, said groove could be connected to said supply duct, at the level of the distal end thereof, via linking means. These could comprise a ring, being free rotatably mounted around the supply duct, and at least one linking arm, being integral with said ring and with said groove.

Furthermore, when said tubular nozzle is connected to said source of respiratory gas by means of said supply duct, said groove extends longitudinally, preferably, at least up to the proximal end of said tubular nozzle, so as to counteract against assembling a member for connecting an external medical device.

Preferably, said groove has a semi-cylindrical shape for enabling that said nozzle is properly maintained, when the supply duct is connected to the latter. In this latter case, the tubular nozzle and the groove it is housed in, are substantially concentric.

According to another embodiment according to the present method, system and device, said tubular nozzle comprises:
a projecting side tubular member intended for being connected to said respiratory gas supply duct; and
an annular chamber where said projecting side member and said auxiliary channel open into; and
a groove comprising an opening intended for being crossed by said projecting side member, when said tubular nozzle is connected to said source by means of the supply duct.

Moreover, the distal end of the supply duct could comprise a fastening nut, free rotatable mounted around said duct, being able to cooperate with complementary fastening means arranged at the free end of said projecting side member.

Additional examples and details of the present method, system and device are also described in the following detailed description, drawings, and appended claims.

Various embodiments of the present method, system and device are described in detail in the detailed description and claims below.

Any feature or combination of features described herein are included within the scope of the present method, system and device and in combination whether expressly described provided that the features included in any such combination are not mutually inconsistent as will be apparent from the context of the described features and knowledge of one of ordinary skill in the art. In addition, any feature or combination of features may be specifically excluded from any embodiment of the present method, system and device.

### BRIEF DESCRIPTION OF THE FIGURES

The figures of the appended drawing will better explain how the present method, system and device can be implemented. In these figures, like reference numerals relate to like components.

FIG. 1 is a general view of an example of a ventilatory assistance system, according to the present method, system and device, comprising a ventilatory assistance device and a respiratory gas supply duct. In this FIG., the supply duct is shown connected to the ventilatory assistance device.

FIG. 2 partially illustrates, in a longitudinal schematic section, the ventilatory assistance device of FIG. 1, when it is not connected to the supply duct.

FIG. 3 is a schematic cross-section along line III-III of FIG. 2 of the ventilatory assistance device of FIG. 1.

FIG. 4 shows, in a partial schematic view, the tubular nozzle and the obstacle forming means of the system of FIG. 1, when the supply duct is disconnected from said tubular nozzle.

FIG. 5 shows, in a schematic end view, the tubular nozzle and the obstacle forming means of the system of FIG. 1, when the supply duct is connected to said tubular nozzle.

### DETAILED DESCRIPTION

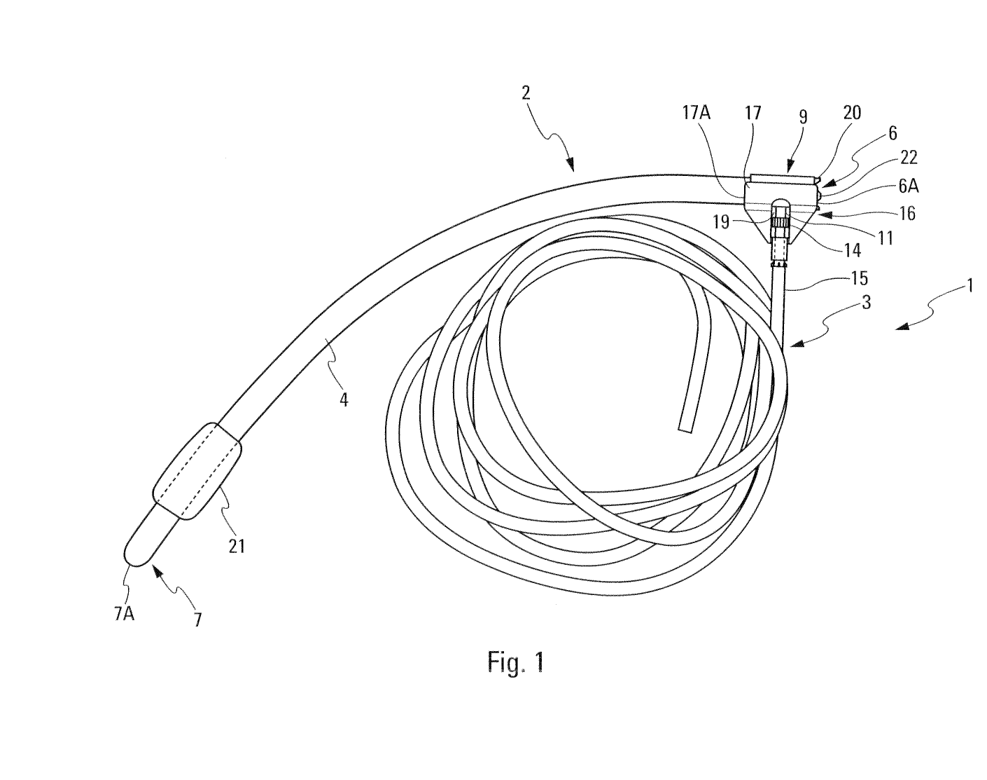

FIG. 1 schematically shows an example of a ventilatory assistance system 1 according to the present method, system and device, comprising a ventilatory assistance device 2 and a respiratory gas supply duct 3 able to be connected to a source of respiratory gas (not shown) for instance a bottle of oxygen.

In this example, the device 2 has the shape of an endotracheal probe with a small balloon, more specifically used in the case of a cardiac arrest in a patient. When this probe is connected by the supply duct 3 to a source of respiratory gas, it forms a passive oxygenation device.

Obviously, the present method, system and device are by no means limited to this example, so that the ventilatory assistance device could then also have the shape of a positive pressure spontaneous ventilation device CPAP used as a passive oxygenation device on a patient in a cardiac arrest condition, a pediatric endotracheal probe, a supraglottic device (for instance sold on the market under the brand KING SYSTEMS, or even COMBITUBE), a gas monitoring probe, an endobronchial probe, a child anatomic intubation probe, etc.

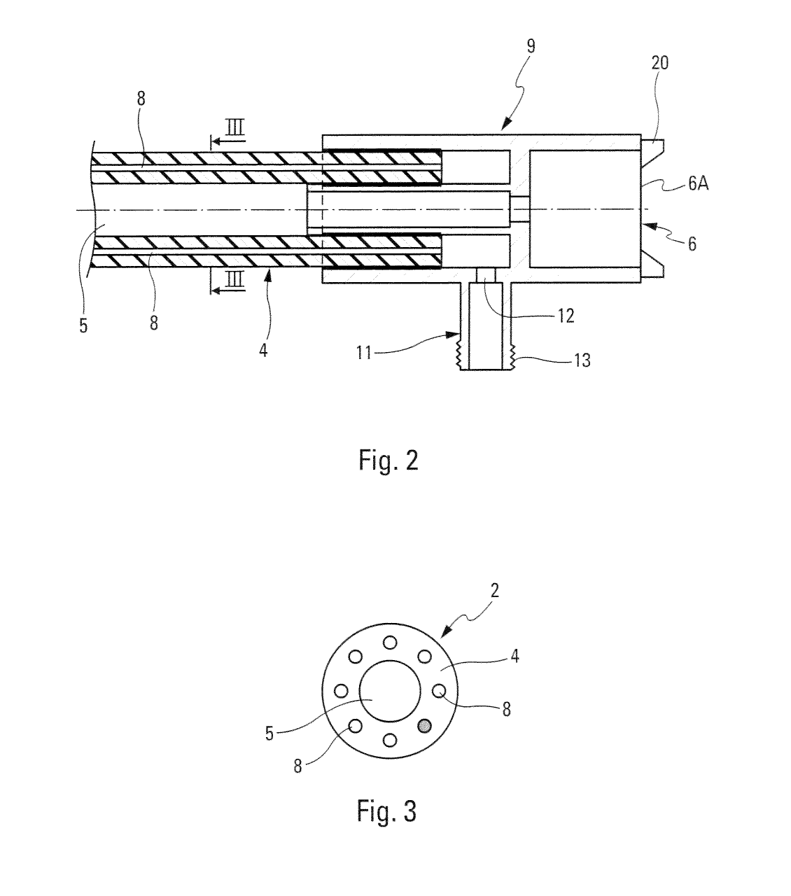

As shown on FIGS. 1 and 2, the device 2 comprises, as known, a flexible or preformed tube 4 (for adapting to the morphology of the patient being managed) delimiting a main channel 5 having a proximal hole 6A and a distal hole 7A, respectively at the proximal and distal ends 6. 7 of said tube 4.

Thus, the main channel 5 is able to provide the passage between the proximal and distal holes (6A, 7A, one of which (the distal hole 7A) and is to be positioned inside the patient's airways and the other one (the proximal hole 6A) is to be positioned outside them. This proximal hole 6A could open into the open air and, in this case, the patient is able to inhale fresh air and to exhale stale air through the main channel 5.

Moreover, in the thickness of the wall of the tube 4, longitudinal auxiliary channels 8 are arranged, extending nearly on the whole length of the tube 4 and intended to be connected, for at least some of them, to a source of pressurized respiratory gas. The longitudinal channels 8 open into the main channel 5, in the vicinity of its distal end 7.

As shown on FIG. 3, the auxiliary channels 8 are arranged regularly around the axis of the tube 4. At least one of the auxiliary channels 8 (represented as a filled circle in FIG. 1) could be dedicated for supplying a medical fluid (for instance a drug).

The tube 4 of the device 2 could be made in any material already used in respiratory probes, for instance made in a polyvinyl chloride, with a possible coating in silicone or in steel allowing high pressure injections.

Moreover, an inflatable small balloon 21 is mounted on the external side wall of the tube 4, in a vicinity of the distal end 7 thereof. When the tube 4 is positioned in the patient's trachea, the inflatable small balloon 21 can, once inflated, provide the tightness between the trachea and the external outline of the tube 4.

The device 2 also comprises a tubular nozzle 9 being integrally mounted, tightly, at the proximal end 6 of the tube 4. The nozzle 9 is, for instance, able to connect the tube 4 to a (not shown) artificial respirator at the level of the proximal end 6 thereof.

The nozzle 9 comprises an internal annular chamber 10, formed in the area of connection of the tube 4 and of the nozzle 9 and intended to be connected to the source of respiratory gas, by means of the supply duct 3.

Once connected to the source of respiratory gas, the annular chamber 10 becomes tight to the ambient external air. In the latter case, the annular chamber 10 is only crossed by respiratory gas originating from said source and the proximal ends of the auxiliary channels 8, opening into the annular chamber 10, are then supplied with respiratory gas.

As shown on FIGS. 1 and 2, the tubular nozzle 9 further comprises a projecting side tubular 11 communicating with the annular chamber 10 through a hole 12 arranged in the side wall of the nozzle 9.

Moreover, the projecting tubular member 11 is adapted for being connected to the supply duct 3. To this end, it comprises a thread 13 on its external side wall, being intended to cooperate with a complementary nut 14 free rotatably mounted on the distal end 15 of the supply duct 3.

According to the present method, system and device, as illustrated in the figures, the system 1 comprises obstacle forming means 16, counteracting against assembling a member for connecting an external medical device, for instance an artificial respirator, to the tubular nozzle 9 of the device 2, when the latter is connected to the source of respiratory gas by means of the supply duct 3.

In the described example, the obstacle forming means 16 are mounted at the distal end 15 of the supply duct 3. Obviously, alternatively, such means could be arranged in the vicinity of the proximal end of the ventilatory assistance device.

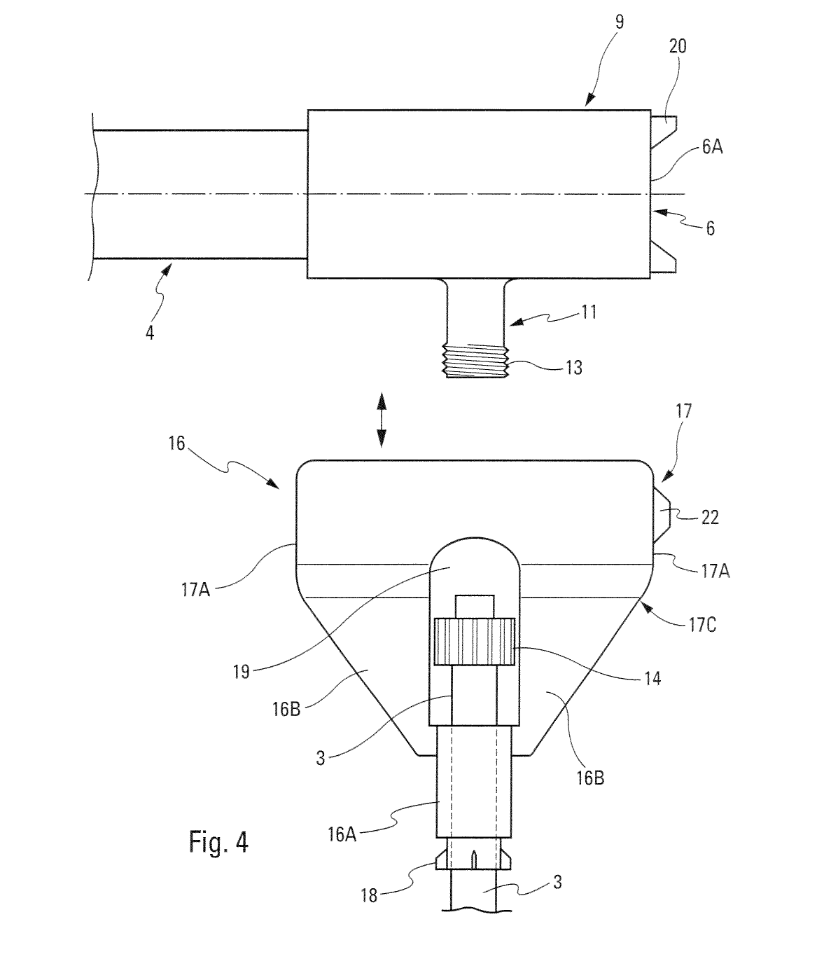

As shown on FIGS. 1, 4 and 5, the obstacle forming means 16 comprise:
a semi-cylindrical groove 17, having the two longitudinal ends 17A being opened. The interior of the groove 17 defines a housing having a shape complementary to that of the tubular nozzle 9;
a ring 16A, free rotatable mounted around the supply duct 3; and
two linking arms 16B rigidly connecting the bottom 17C of the groove 17 to the ring 16A.

Thus, the obstacle forming means 16 are free rotatably mounted around the duct 3, when the latter is not connected to the nozzle 9. Moreover, in this latter case, they can also move along the longitudinal direction of the duct 3. However, such a longitudinal move is limited, on the distal side, by abutments 18 under the form of a fin arranged on the supply duct 3 and, on the proximal side, by the nut 14.

Furthermore, in the bottom 17C of the groove 17, an opening 19 is arranged allowing the projecting side member 11 to cross through it upon the latter being connected to the supply duct 3.

Moreover, the dimensions of the external cross-section of the nozzle 9 are advantageously lower than those of the inner cross-section of the groove 17, so that the tubular nozzle 9 is able to be readily received inside the latter.

Thus, when the side member 11 is fastened to the duct 3 by means of the nut 14, after having crossed the opening 19, the nozzle 9 is housed inside the groove 17.

In such an arrangement, the side wall 17B of the groove 17 covers:
in the direction of the length (defined according to the longitudinal direction of the nozzle 9), the whole nozzle 9. Advantageously, the side wall extends longitudinally beyond the distal end 6 of the nozzle 9; and
in the direction of the height (defined according to a direction being orthogonal with respect to the longitudinal direction), one part of the nozzle 9.

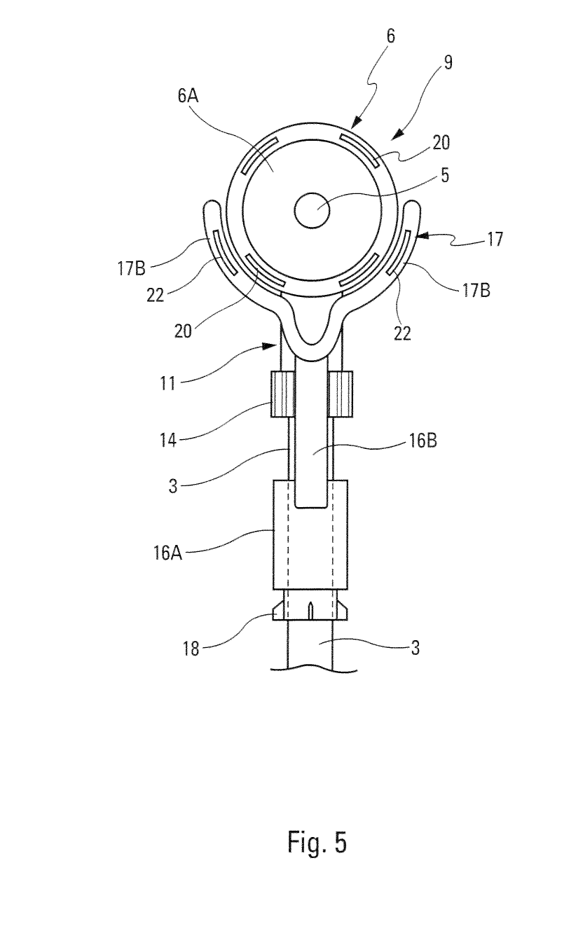

As shown on FIG. 5, the groove 17, once arranged on the tubular nozzle 9, results in an artificial and reversible increase of the diameter of the external cross-section of the nozzle 9.

Thus, connecting a member for connecting a medical device, such as an artificial respirator, cannot be achieved, as the cross-section of the member for connecting the external device is intended for cooperating, with an adjustment, with that of the groove-free tubular nozzle 9.

Furthermore, the tubular nozzle 9 also comprises fins 20, arranged on the circumference of the proximal hole 6A, in the continuation of the side wall of the nozzle 9, so to prevent the latter from being obstructed.

Similarly, the obstacle forming means 16 comprise fins 22, for instance arranged at the proximal longitudinal end of the groove 17, so as to avoid the proximal hole 6A from being obstructed when said nozzle 9 is connected to said source of respiratory gas by means of the duct 3.

It is understood that such fins could be arranged either only on the obstacle forming means, or, on the contrary, only on the tubular nozzle.

Should a cardiac arrest occur in a patient, the auxiliary nursing staff introduces, in a first step, a part of the tube 4 in the patient's trachea.

The supply duct 3 is afterwards connected to the projecting side tubular member 11, so as to connect the tubular nozzle 9 to the source of respiratory gas and thereby supply respiratory gas to the appropriate auxiliary channels 8. In such an arrangement, the groove 17 of the obstacle forming means partially surrounds the tubular nozzle 9, so that no artificial respirator could be connected to the latter upon the continuous insufflation of respiratory gas.

The patient's ventilation, achieved through the continuous insufflation of respiratory gas of the system 1 being combined to compressions and decompressions carried out by the auxiliary nursing staff generating a positive intra thoracic pressure (during the compressions) and a negative intra thoracic pressure (during the decompressions) allowing to achieve a better hemodynamics, is thereby fully secured.

There is no risk of an overpressure occurring inside said patient's airways upon the continuous insufflation of respiratory gas.

After the heart is working again, the supply duct 3 is disconnected from the side tubular member 11, so as to allow an artificial respirator to be connected on the tubular nozzle 9 of the device 2.

The present method, system and device have been described in relation with an example of obstacle forming means having, more specifically, the shape of a semi-cylindrical groove and being mounted at the distal end of the supply duct. It is understood that the present method, system and device are not limited to this embodiment, but also comprises any other appropriate shape of obstacle forming means intended for preventing an external medical device to be connected to the tubular nozzle, when the latter is connected to a source of respiratory gas through the supply duct. Moreover, although being particularly adapted to the case of cardiac arrests, the present method, system and device are by no means restricted to such an application.

1. A ventilatory assistance system comprising:
a ventilatory assistance device comprising:
a tubular body, forming a main channel, configured for connecting, through its distal end, to an airway of a patient so that the main channel connects, outside, the patient's breathing system;
at least one auxiliary channel opening into the main channel upstream of the distal end of the latter; and
a tubular nozzle integrally mounted, tightly, at the proximal end of the tubular body; and
a respiratory gas supply duct configured for connecting said tubular nozzle to a source of respiratory gas, so as to supply respiratory gas to said auxiliary channel,
wherein said system comprises obstacle forming device, counteracting against assembling a connecting member of an external medical device to said tubular nozzle, when the latter is connected to said source of respiratory gas by means of said supply duct.

2. The ventilatory assistance system according to claim 1, wherein said obstacle forming device is mounted at the distal end of said supply duct.

3. The ventilatory assistance system according to claim 2, wherein said obstacle forming device comprises an opened groove defining a housing able to receive at least one part of said tubular nozzle, when the latter is connected to said source of respiratory gas by said supply duct.

4. The ventilatory assistance system according to claim 3, wherein said groove is connected to said supply duct, at the level of its distal end, by linking means.

5. The ventilatory assistance system according to claim 4, wherein said linking means comprise a ring, being free rotatably mounted around the supply duct, and at least one linking arm, integral with said ring and with said groove.

6. The ventilatory assistance system according to claim 3, wherein when said tubular nozzle is connected to said source of respiratory gas by means of said supply duct, said groove longitudinally extends at least up to the proximal end of said tubular nozzle, so as to counteract against assembling a connecting member of an external medical device.

7. The ventilatory assistance system according to claim 3, wherein said groove has a semi-cylindrical shape.

8. The ventilatory assistance system according to claim 3, wherein said tubular nozzle comprises:
a projecting side tubular member intended for being connected to said respiratory gas supply duct; and
an annular chamber where said projecting side member and said auxiliary channel open into; and
wherein the groove comprises an opening intended for being crossed by said projecting side tubular member, when said tubular nozzle is connected to said source by means of the supply duct.

9. The ventilatory assistance system according to claim 8, wherein the distal end of the supply duct comprises a fastening nut, being free rotatable mounted around said duct, being able to cooperate with complementary fastening means arranged at the free end of said projecting side member.

10. The ventilatory assistance system according to claim 1, wherein said obstacle forming means comprise at least one fin for preventing any obstruction of the proximal end of said tubular nozzle, when the latter is connected to said source of respiratory gas by means of said supply duct.

11. The ventilatory assistance system according to claim 1, wherein said nozzle comprises, at its proximal end, at least one fin, for preventing any obstruction of the latter.
